(19) United States
(12) Patent Application Publication
(10) Pub. No.: US 2024/0016717 A1
(43) Pub. Date: Jan. 18, 2024

(54) COMPOSITION COMPRISING GLYCOLIPID AND SALICYLIC ACID DERIVATIVE

(71) Applicant: L OREAL, PARIS (FR)

(72) Inventors: Masanori ORITA, Kawasaki-shi (JP); Koji ENDO, Kawasaki-shi (JP); Hiroaki KAGA, Kawasaki-shi (JP)

(21) Appl. No.: 18/042,541

(22) PCT Filed: Aug. 27, 2021
(86) PCT No.: PCT/JP2021/032564

§ 371 Date: Feb. 22, 2023

(30) Foreign Application Priority Data
Aug. 28, 2020 (JP) 2020-144452
Sep. 25, 2020 (FR) 2009756

(51) Int. Cl. A61K 8/60; A61K 8/368; A61Q 19/10

(52) U.S. Cl. CPC A61K 8/60; A61K 8/368; A61Q 19/10; A61K 2800/5922

(57) ABSTRACT

The present invention relates to a composition comprising (a) at least one glycolipid; and (b) at least one salicylic acid derivative represented by formula (I). The composition according to the present invention can provide an effect of removing or decreasing keratinous plugs in keratinous substances, such as skin.

### TECHNICAL FIELD

The present invention relates to a composition comprising at least one glycolipid and at least one salicylic acid derivative, preferably a cosmetic composition comprising the same.

### BACKGROUND ART

Keratinous plugs can be observed inside pores on the face, such as pores around the nose. When the keratinous plugs are left without being removed inside the pores, they grow inside the pores and they could physically expand the pores. This may deteriorate the aesthetic properties of the face. Therefore, a variety of techniques for removing or decreasing keratinous plugs have been conventionally developed.

Therefore, there is a need to develop a composition which can remove or decrease keratinous plugs in the skin, in particular facial skin, in order to make pores smaller and attain a good appearance of the skin.

### DISCLOSURE OF INVENTION

An objective of the present invention is to provide a composition which can remove or decrease keratinous plugs in keratinous substances, such as skin.

The above objective of the present invention can be achieved by a composition comprising:
(a) at least one glycolipid; and
(b) at least one salicylic acid derivative of formula (I):

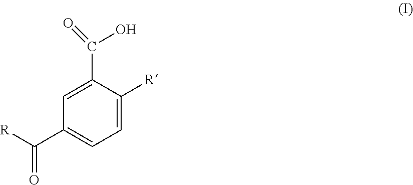

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

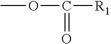

in which R₁ denotes a linear or branched, saturated or unsaturated aliphatic chain containing 1 to 18 carbon atoms;
and also salts thereof derived from an inorganic or organic base.

The (a) glycolipid may be selected from autos and sophorolipids.

The (b) salicylic acid derivative may be represented by formula (I) in which the radical R denotes a linear, branched or cyclic, saturated aliphatic chain containing from 2 to 22 carbon atoms.

The (b) salicylic acid derivative may be represented by formula (I) in which the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

The (b) salicylic acid derivative may be represented by formula (I) in which R′ denotes a hydroxyl group.

The amount of the (a) glycolipid(s) may be 0.1% by weight or more, preferably 0.2% by weight or more, more preferably 0.5% by weight or more, and in particular 0.8% by weight or more, and is 10% by weight or less, preferably 7% by weight or less, more preferably 5% by weight or less, and in particular 4% by weight or less, relative to the total weight of the composition.

The amount of the (b) salicylic acid derivative(s) may be 0.01% by weight or more, preferably 0.05% by weight or more, more preferably 0.1% by weight or more, and in particular 0.15% by weight or more, and is 5% by weight or less, preferably 3% by weight or less, more preferably 1% by weight or less, and in particular 0.5% by weight or less, relative to the total weight of the composition.

The weight ratio of the (a) glycolipid(s) to the (b) salicylic acid derivative(s) in the composition may be 1 or more, preferably 2 or more, more preferably 3 or more, even more preferably 4 or more, and in particular 5 or more.

The composition according to the present invention may be in the form of a solution.

The composition may further include water in an amount of 60% by weight or more, preferably 70% by weight or more, and more preferably 80% by weight or more, relative to the total weight of the composition.

The composition may be a cosmetic composition for caring for, conditioning, and/or cleansing keratinous substances, such as skin.

The present invention also relates to a non-therapeutic cosmetic process for caring for, conditioning, and/or cleansing keratinous substances, such as skin, comprising:
applying onto the keratinous substances the composition according to the present invention.

The present invention also relates to a use of a combination of the (a) glycolipid and the (b) salicylic acid derivative, for caring for, conditioning, and/or cleansing keratinous substances, such as skin.

### BEST MODE FOR CARRYING OUT THE INVENTION

After diligent research, the inventors have surprisingly discovered that a composition comprising (a) at least one glycolipid and (b) at least one salicylic acid derivative having the specific structure of formula (I) can remove or decrease keratinous plugs in the skin, thus completing the present invention.

Thus, the composition according to the present invention is a composition comprising:
(a) at least one glycolipid; and
(b) at least one salicylic acid derivative of formula (I):

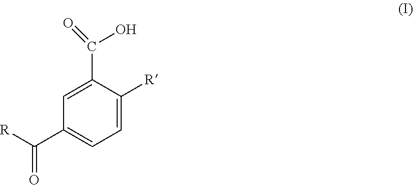

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

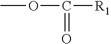

in which R₁ denotes a linear or branched, saturated or unsaturated aliphatic chain containing 1 to 18 carbon atoms;
and also salts thereof derived from an inorganic or organic base.

Hereinafter, the composition, process, and use according to the present invention will be explained in a more detailed manner.

[Composition]

The composition may be a cosmetic composition, preferably a cosmetic composition for a keratinous substance, and more preferably a cosmetic composition for caring for, conditioning, and/or cleansing a keratinous substance. The keratinous substance here means a material containing keratin as a main constituent element, and examples thereof include the skin, scalp, lips, and the like.

The form of the composition according to the present invention is not particularly limited. In general, the composition according to the present invention is liquid at room temperature (25° C.) and atmospheric pressure (10⁵ Pa). The composition may take various forms, such as a solution, an aqueous solution, a lotion, a milky lotion, a cream, a gel, a liquid gel, a paste, a serum, a suspension, a dispersion, a fluid, a milk, an emulsion (O/W or W/O form), or the like. It is preferable that the composition according to the present invention be in the form of an aqueous solution or gel.

The composition according to the present invention may preferably be used as a cosmetic composition, in particular a rinse-off type cosmetic composition. The composition according to the present invention may be intended for application onto a keratinous substance, preferably skin, and in particular facial skin.

The composition according to the present invention comprises (a) at least one glycolipid; and (b) at least one salicylic acid derivative according to formula (I). The ingredients in the composition will be described in a detailed manner below.

(Glycolipid)

The composition according to the present invention comprises at least one (a) glycolipid. If two or more (a) glycolipids are used, they may be the same or different.

The (a) glycolipid may suitably be selected from rhamnolipids, glucolipids, trehalolipids, and mixtures thereof. Each will now be described in more detail below.

Rhamnolipids

These glycolipids include a rhamnose moiety, and can be represented by general formula (I):

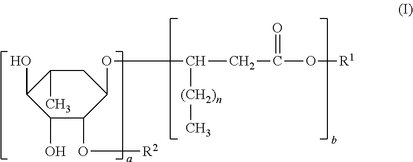

where
a is 1 or 2,
b is 1 or 2,
n is 4 to 10, preferably 6,
R¹ is H or a cation, preferably H, or a monovalent solubilizing cation,
R² is H or the group

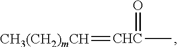

preferably H,
m is 4 to 10, and
the values of m and n need not be the same at each occurrence.

Rhamnolipids can be produced by bacterial fermentation. This is inherently advantageous in that products of bacterial fermentation can generally be derived from renewable raw materials and are likely to be biodegradable after use. Another advantage of the surfactants of formula (I) is that they can be produced as a by-product of enzyme manufacture.

Rhamnolipids can be produced by bacteria of the genus Pseudomonas. The bacterial fermentation typically utilizes as substrates a sugar or glycerol or an alkane or mixtures thereof.

Appropriate fermentation methods are reviewed in D. Haferburg, R. Hommel, R. Claus and H. P. Kleber in Adv Biochem. Eng./Biotechnol. (1986) 33, 53-90 and by F. Wagner, H. Bock and A. Kretschmar in Fermentation (ed. R. M. Lafferty) (1981), 181-192, Springer Verlag, Vienna.

Any sample of rhamnolipids will generally contain a variety of individual compounds within general formula (I). The proportions of individual compounds are governed by the microorganism species, and the particular strain employed for fermentation, the substrate materials supplied to the fermentation, and other fermentation conditions.

Bacterial fermentation generally produces compounds in which R¹ is hydrogen or a solubilizing cation. Such compounds can undergo conversion between the salt and the acid forms in aqueous solution, according to the pH of the solution. Common solubilizing cations are alkali metal, ammonium and alkanolamine.

As the rhamnolipids, for example, that sold under the name of RHEANCE One from EVONIK can be used.

Glucolipids

A second class of the glycolipid in accordance with the present invention comprises glucolipids, which include a glucose moiety and can be represented by general formula (II):

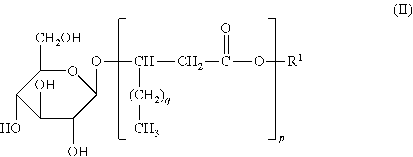

where
R¹ is H or a cation,
p is 1 to 4; and
q is 4 to 10, preferably 6.

Glucolipids can be produced by the bacterium Alcaligenes Sp.MM1. Appropriate fermentation methods are reviewed by M. Schmidt in his Ph.D. thesis (1990), Technical University of Braunschweig, and by Schulz et al. (1991) Z. Naturforsch 46C 197-203. The glucolipids are recovered from the fermentation broth via solvent extraction using ethyl ether or a mixture of either dichloromethane:methanol or chloroform:methanol

Sophorolipids

A third class of the glycolipid in accordance with the present invention comprises sophorolipids, which include a sophorose moiety and can be represented by general formula (III):

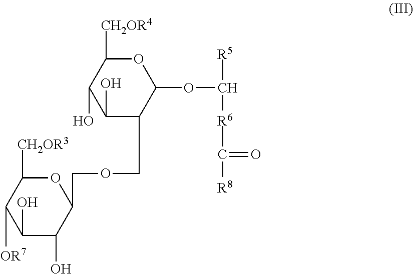

where
R³ and R⁴ are individually H or an acetyl group,
R⁵ is a saturated or unsaturated, hydroxylated or non-hydroxylated hydrocarbon group having 1 to 9 carbon atoms, preferably being a methyl group,
R⁶ is a saturated or unsaturated hydroxylated or non-hydroxylated hydrocarbon group having 1 to 19 carbon atoms,
with the proviso that the total number of carbon atoms in groups R⁵ and R⁶ does not exceed 20 and is preferably from 14 to 18.

The sophorolipids may be incorporated into detergent compositions of the present invention as either the open chain free acid form, where R⁷ is H and R⁸ is OH, or in its lactone form, where a lactone ring is formed between R⁷ and R⁸ as shown by general formula (IV):

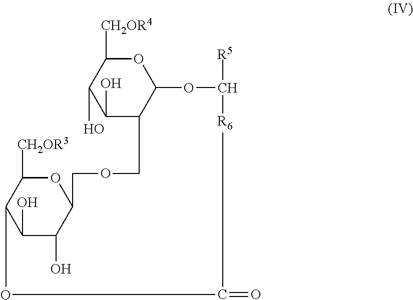

where
R³, R⁴, and R⁶ are as defined above,
with the proviso that at least one of R³ and R⁴ is an acetyl group.

Sophorolipids can be produced by yeast cells, for example Torulopsis apicola and Torulopsis bombicola. The fermentation process typically utilizes sugars and alkanes as substrates. Appropriate fermentation methods are reviewed in A. P. Tulloch, J. F. T. Spencer and R A. J. Gorin, Can. J Chem (1962) 40 1326 and U. Gobbert, S. Lang and F. Wagner, Biotechnology Letters (1984) 6 (4), 225. The resultant product is a mixture of various open-chain sophorolipids and sophorolipid lactones, which may be utilized as mixtures, or the required form can be isolated. When the glycolipid comprises sophorolipids, the weight ratio of sophorolipids to additional surfactants is preferably in the range 4:1 to 3:2 and is more preferably 4:1.

As the sophorolipids, for example, that sold under the name of SOPHOLIANCE S from GIVAUDAN and that sold under the name of BioToLife from BASF can be used.

Trehalolipids

A fourth class of the glycolipid in accordance with the present invention comprises trehalolipids, which include a trehalose moiety and can be represented by general formula (V):

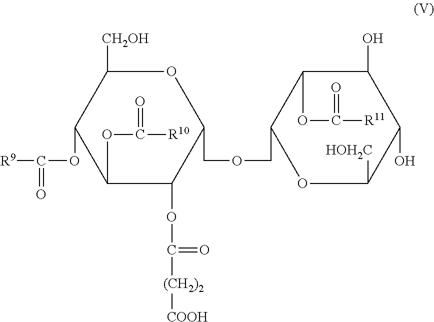

where
R⁹, R¹⁰, and R¹¹ are individually a saturated or unsaturated, hydroxylated or non-hydroxylated hydrocarbon of 5 to 13 carbon atoms.

Trehalolipids can be produced by bacteria fermentation using the marine bacterium Arthrobacter sp. Ek 1 or the fresh water bacterium Rhodococcus erythropolis. Appropriate fermentation methods are provided by Ishigami et al. (1987) J. Jpn Oil Chem Soc 36 847-851, Schultz et al. (1991), Z. Naturforsch 46C 197-203; and Passeri et al. (1991) Z. Naturforsch 46C 204-209.

Cellobioselipids

A fifth class of the glycolipid in accordance with the present invention comprises cellobioselipids, which include a cellobiose moiety and can be represented by general formula (VI):

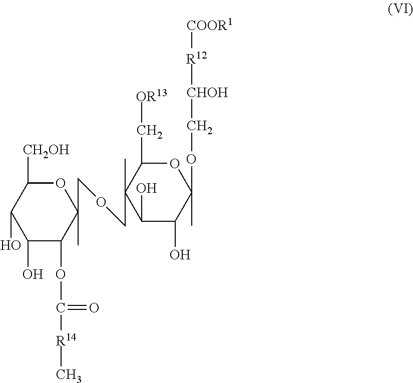

where
R¹ is H or a cation,
R¹² is a saturated or non-saturated, hydroxylated or non-hydroxylated hydrocarbon having 9 to 15 carbon atoms, preferably 13 carbon atoms,
R¹³ is H or an acetyl group; R¹⁴ is a saturated or non-saturated, hydroxylated or non-hydroxylated hydrocarbon having 4 to 16 carbon atoms.

Cellobioselipids can be produced by fungi cells from the genus Ustilago. Appropriate fermentation methods are provided by Frautz, Lang and Wagner (1986) Biotech Letts 8 757-762.

It may be preferable that the (a) glycolipid be selected from rhamnolipids and sophorolipids.

The (a) glycolipid(s) may be present in an amount of 0.1% by weight or more, preferably 0.2% by weight or more, more preferably 0.5% by weight or more, and in particular 0.8% by weight or more, and may be present in an amount of 10% by weight or less, preferably 7% by weight or less, more preferably 5% by weight or less, and in particular 4% by weight or less, relative to the total weight of the composition.

(Salicylic Acid Derivative)

The composition according to the present invention comprises (b) at least one salicylic acid derivative according to formula (I). A single type of the salicylic acid derivative may be used, but two or more different types of the salicylic acid derivatives may be used in combination.

The salicylic acid derivatives in accordance with the present invention correspond to formula (I):

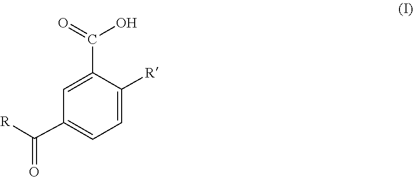

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

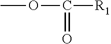

in which R₁ denotes a linear or branched, saturated or unsaturated aliphatic chain containing 1 to 18 carbon atoms;
and also salts thereof derived from an inorganic or organic base.

Preferably, the R radical in formula (I) denotes a linear, branched or cyclic, saturated aliphatic chain containing from 3 to 11 carbon atoms; an unsaturated chain containing from 3 to 17 carbon atoms and comprising one or more conjugated or unconjugated double bonds; it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one preferred embodiment, the R radical in formula (I) denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

Preferentially, R′ in formula (I) is a hydroxyl group or an ester group of formula:

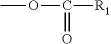

in which R₁ denotes a radical —(CH₂)ₙ—CH₃ where n is a number ranging from 0 to 14.

The salicylic acid derivatives that are more particularly preferred are those according to formula (I) in which the R radical is a C₃-C₁₀ alkyl group and/or R′ denotes hydroxyl.

Other particularly advantageous compounds are those in which R represents a chain derived from caprylic, linoleic, linolenic or oleic acid.

Another group of particularly preferred salicylic acid derivatives is constituted of compounds in which the R radical denotes a C₃-C₁₀ alkyl group bearing a carboxyl function in free form or esterified with a lower alcohol containing from 1 to 6 carbon atoms and R′ denotes hydroxyl.

The salicylic acid derivatives of formula (I) that may be used according to the present invention are in particular described in U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A-570,230.

Among the particularly preferred salicylic acid derivatives of formula (I), mention may be made of 5-n-octanoylsalicylic acid (or capryloyl salicylic acid); 5-n-decanoylsalicylic acid; 5-n-dodecanoylsalicylic acid; 5-n-heptyloxysalicylic acid, and the corresponding salts thereof. The derivative in question is preferably 5-n-octanoylsalicylic acid.

For the purposes of the present invention, the salts of the salicylic acid derivatives are also considered. As the salts derived from inorganic bases, mention may particularly be made of those derived from alkali metal or alkaline-earth metal hydroxylated bases, for instance sodium hydroxide or potassium hydroxide, and ammonia. As regards the salts derived from the organic bases, mention may particularly be made of those derived from bases of amine or alkanolamine type.

The (b) salicylic acid derivative(s) may be present in an amount of 0.01% by weight or more, preferably 0.05% by weight or more, more preferably 0.1% by weight or more, and in particular 0.15% by weight or more, and may be present in an amount of 5% by weight or less, preferably 3% by weight or less, more preferably 1% by weight or less, and in particular 0.5% by weight or less, relative to the total weight of the composition.

According to one preferred embodiment of the present invention, the weight ratio of the (a) glycolipid(s) to the (b) salicylic acid derivative(s) in the composition can be 1 or more, preferably 2 or more, more preferably 3 or more, even more preferably 4 or more, and in particular 5 or more. In general, the weight ratio of the (a) glycolipid(s) to the (b) salicylic acid derivative(s) in the composition is 50 or less, preferably 30 or less, and more preferably 20 or less.

In one preferred embodiment of the present invention, the composition comprises:
(a) at least one biosurfactant selected from rhamnolipids and sophorolipids; and
(b) at least one salicylic acid derivative according to formula (I):

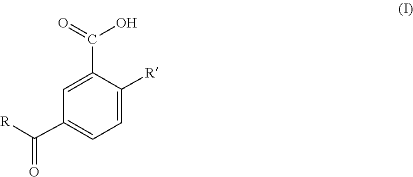

in which
the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms, and R′ is a hydroxyl group.

(Amphoteric Surfactant)

The composition according to the present invention may comprise at least one amphoteric surfactant. Two or more amphoteric surfactants may be used in combination.

The amphoteric or zwitterionic surfactants can be, for example (non-limiting list), amine derivatives such as aliphatic secondary or tertiary amine, and optionally quaternized amine derivatives, in which the aliphatic radical is a linear or branched chain including 8 to 22 carbon atoms and containing at least one water-solubilizing anionic group (for example, carboxylate, sulphonate, sulphate, phosphate, or phosphonate).

The amphoteric surfactant may preferably be selected from the group consisting of betaines and amidoaminecarboxylated derivatives.

It is preferable that the amphoteric surfactant be selected from betaine-type surfactants.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄)alkylbetaines, (C₈-C₂₄)alkylamido(C₁-C₈)alkylbetaines, sulphobetaines, and (C₈-C₂₄)alkylamido(C₁-C₈)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, sulphobetaines, and phosphobetaines.

Non-limiting examples that may be mentioned include the compounds classified in the CTFA International Cosmetic Ingredient Dictionary & Handbook, 15th Edition, 2014, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

The amount of the amphoteric surfactant(s) in the composition according to the present invention may be 0.1% by weight or more, preferably 0.5% by weight or more, more preferably 1% by weight or more, and even more preferably 2% by weight or more; and 20% by weight or less, preferably 15% by weight or less, more preferably 10% by weight or less, and even more preferably 5% by weight or less, relative to the total weight of the composition.

(Cosmetically Acceptable Hydrophilic Organic Solvent)

The composition according to the present invention may comprise at least one cosmetically acceptable hydrophilic organic solvent. The term “hydrophilic” here means substances having a solubility of at least 1 g/L, preferably at least 10 g/L, and more preferably at least 100 g/L, in water at room temperature (25° C.) and atmospheric pressure (10⁵ Pa). The cosmetically acceptable hydrophilic organic solvent(s) may include, for example, substantially linear or branched lower mono-alcohols having from 1 to 8 carbon atoms, such as ethanol, propanol, butanol, isopropanol, and isobutanol; aromatic alcohols, such as benzyl alcohol and phenylethyl alcohol; polyols or polyol ethers, such as propylene glycol, dipropylene glycol, isoprene glycol, butylene glycol, glycerine, propanediol, caprylyl glycol, sorbitol, ethylene glycol monomethyl, monoethyl and monobutyl ethers, propylene glycol ethers, such as propylene glycol monomethylether, diethylene glycol alkyl ethers, such as diethylene glycol monoethylether or monobutylether; polyethylene glycols, such as PEG-4, PEG-6, and PEG-8, and their derivatives, and a combination thereof.

The amount of the cosmetically acceptable hydrophilic organic solvent(s) in the composition according to the present invention may be from 1% by weight or more, preferably 2% by weight or more, and more preferably 5% by weight or more, and may be 25% by weight or less, preferably 20% by weight or less, and more preferably 15% by weight or less, relative to the total weight of the composition.

(pH Adjusting Agent)

The pH of the composition according to the present invention may be adjusted to the desired value using at least one pH adjusting agent, such as an acidifying or a basifying agent, for example, which are commonly used in cosmetic products.

The pH of the composition according to the present invention may be 9.0 or less, more preferably 8.5 or less, and even more preferably 8.0 or less, and be 5.0 or more, more preferably 5.5 or more, and even more preferably 6.0 or more.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide; quaternary ammonium hydroxides and guanidinium hydroxide; alkali metal silicates, such as sodium metasilicates; amino acids, preferably basic amino acids, such as arginine, lysine, ornithine, citrulline and histidine; carbonates and bicarbonates, particularly of a primary amine, secondary amine or tertiary amine, of an alkali metal or alkaline-earth metal, or of ammonium; and the compounds of the following formula:

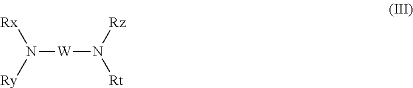

in which
W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2-propanol, spermine and spermidine.

The pH adjusting agent(s) may be used in an amount ranging from 0.001% to 10% by weight, preferably from 0.01% to 5% by weight, and more preferably from 0.1% to 3% by weight, relative to the total weight of the composition.

(Water)

The composition according to the present invention preferably includes water.

The amount of water in the composition according to the present invention may be 60% by weight or more, preferably 70% by weight or more, and more preferably 80% by weight or more; and may be 99% or less, relative to the total weight of the composition.

(Optional Additives)

The composition according to the present invention may also comprise any optional additive(s) usually used in the field of cosmetics, chosen, for example, from anionic, cationic, nonionic or amphoteric polymers, anionic, cationic, nonionic or amphoteric surfactants, oils, hydrophobic organic solvents, gums, resins, thickeners, dispersants, antioxidants, buffers, organic or inorganic fillers, preserving agents, such as phenoxyethanol, fragrances, neutralizers, antiseptics, UV-screening agents, cosmetic active agents such as vitamins, moisturizers, emollients or collagen-protecting agents, and mixtures thereof.

As the antiseptics, those generally used for cosmetics can be used. As the specific examples of the antiseptics, mention can be made of, for example, ethanol, triclosan, piroctone olamine, triclocarban, paraben, acrinol, alkyldiaminoglycine or a salt thereof, povidone iodine, potassium iodide, iodine, 1,2-pentanediol, halocarban, 3,4,4-trichlorocarbanilide, triethyl citrate, resorcin, hexachlorophene, silver-supported zeolite, silver-supported silica, gluconic acid, benzoic acid, sodium benzoate, undecylene acid, salicylic acid, sorbic acid or a salt thereof, dehydroacetic acid or a salt thereof, methyl paraoxybenzoate, ethyl paraoxybenzoate, propyl paraoxybenzoate, isopropyl paraoxybenzoate, butyl paraoxybenzoate, isobutyl paraoxybenzoate, benzyl paraoxybenzoate; phenols such as isopropylmethylphenol, chlorhexidine gluconate, cresol, timole, parachlorophenol, phenylethyl alcohol, phenylphenol, sodium phenylphenol, sodium phenoxyethanol, phenoxydiglycol, phenol, benzyl alcohol; tertiary ammonium salt compounds such as stearyltrimethylammonium chloride, cetyltrimethylammonium chloride, cetylpyridinium chloride, benzalconium chloride, benzethonium chloride, methylbenzethonium chloride, lauryltrimethylammonium chloride, alkylisoquinolium bromide, domiphenone bromide; plant extracts such as tea extract, hinokithiol, apple extract; chloramine T, chlorhexidine, benzoic acidion, and mixtures thereof.

The antiseptic(s) may be used in an amount ranging from 0.001% to 10% by weight, preferably from 0.01% to 5% by weight, and more preferably from 0.1% to 3% by weight, relative to the total weight of the composition.

The composition according to the present invention can be prepared by mixing the above-described essential and optional ingredients in a conventional manner. In the case that at least one of the above ingredients is solid at room temperature, the ingredient can be heated until it is dissolved. It is possible to further comprise mixing any of the optional ingredients and heating the composition until an ingredient is dissolved.

[Cosmetic Process and Use]

The present invention also relates to a non-therapeutic method or process, preferably a cosmetic method or process, and more preferably a cosmetic method or process for caring for, conditioning, and/or cleansing keratinous substances, such as skin, scalp, lips, in particular facial skin, comprising:
applying onto the keratinous substance a composition comprising:
(a) at least one glycolipid; and
(b) at least one salicylic acid derivative of formula (I) above.

The present invention also relates to a use of a combination of
(a) at least one glycolipid; and
(b) at least one salicylic acid derivative of formula (I) above, for caring for, conditioning,
and/or cleansing keratinous substances, such as skin, scalp, lips, in particular facial skin.

The composition is generally applied on a keratinous substance, such as skin, with the hands or an applicator. The present invention may comprise an optional step of rinsing the composition from the keratinous substance after it is applied.

The same explanations for the composition, (a) glycolipid and the (b) salicylic acid derivative as the composition according to the present invention above can be applied to the composition, (a) glycolipid and the (b) salicylic acid derivative for the method, process, and use inventions. The composition used in the process and use according to the present invention may include any of the optional ingredients as explained above for the composition according to the present invention.

### EXAMPLES

The present invention will be described in more detail by way of examples which however should not be construed as limiting the scope of the present invention.

### Examples 1 and 2 and Comparative Examples 1 to 4

Homogeneous solution compositions according to Examples 1 and 2 and Comparative Examples 1 to 4 were prepared by mixing the ingredients as listed in Table 1 with a magnetic stirrer. The 45 numerical values for the amounts of the ingredients shown in Table 1 are all based on “% by weight” as active raw materials. Sophorolipids were obtained from BASF (trade name: BioToLife), and Rhamnolipids were obtained from EVONIK (trade name: RHEANCE One).

[Evaluation]

The effects of each of the compositions on keratinous plugs were assessed as below. Keratinous plugs were obtained by carefully removing them from the nose of men aged in their 30s with a keratinous plug extractor loop. A keratinous plug was gently placed on a slide glass. 20 μL of each compositions was dropped on it while it was recorded on a video microscope, Keyence VHX-5000. The appearances of the keratinous plug were compared between before the composition was applied and 3 minutes after the composition was applied. The more swollen or dispersed the keratinous plug was observed after the application, the better the effect of removing or decreasing keratinous plugs could be recognized. This is because more swollen or dispersed keratinous plugs can be easily removed from keratinous substances, such as skin. Based on the observation, keratinous plug swelling and dispersion efficacies were evaluated with score criteria as below.
0=no obvious change was observed.
1=only a surface of the keratinous plug was swollen.
2=the entirety of the keratinous plug was swollen.
3=the entirety of the keratinous plug was swollen and its volume became approximately twice its original volume or larger.
4=the entirety of the keratinous plug was swollen and its volume became approximately twice its original volume or larger, and a part of the keratinous plug was broken down and dispersed in the solution.

The measurement was repeated 5 times and the average score was calculated. The results are summarized in Table 1 below.

TABLE 1

| | | | Comp. | Comp. | Comp. | Comp. |
|---|---|---|---|---|---|---|
| Ingredients | Ex. 1 | Ex. 2 | Ex. 1 | Ex. 2 | Ex. 3 | Ex. 4 |
| Water | QS 100 | QS 100 | QS 100 | QS 100 | QS 100 | QS 100 |
| Potassium | QS pH7 | QS pH7 | QS pH7 | QS pH7 | QS pH7 | QS pH7 |
| Hydroxide | | | | | | |
| Sophorolipids | 1 | — | 1 | — | 1 | — |
| Rhamnolipids | — | 1 | — | 1 | — | 1 |
| Capryloyl | 0.2 | 0.2 | — | — | — | — |
| Salicylic Acid | | | | | | |
| Salicylic Acid | — | — | — | — | 0.2 | 0.2 |
| Phenoxyethanol | 0.3 | 0.3 | 0.3 | 0.3 | 0.3 | 0.3 |
| Evaluation | | | | | | |
| Average Score | 2.8 | 3.2 | 1.4 | 1 | 2.2 | 1.2 |
| (n = 5) | | | | | | |

As can be seen from Table 1, the compositions according to Examples 1 and 2, which include the specific combination of the ingredients of the (a) glycolipid and the (b) salicylic acid derivative of the present invention, showed higher average scores than those of Comparative Examples 1 to 4 which do not include the specific combination of the (a) glycolipid and the (b) salicylic acid derivative of the present invention. This result means that the compositions according to Examples 1 and 2 exhibited an improved effect of removing or decreasing keratinous plugs.

Therefore, it can be said that the composition according to the present invention enables reduction of the size of pores on keratinous substances by removing or decreasing keratinous plugs in the pores. Accordingly, it can be concluded that the composition according to the present invention is very suitable as cosmetic compositions for caring for, conditioning, and/or cleansing keratinous substances, since it can improve the aesthetics of the keratinous substances.

### Example 3

A homogeneous solution composition according to Example 3 was also prepared, which has a preferred formulation for cosmetic products, by mixing the ingredients shown in Table 2 below. The numerical values for the amounts of the ingredients shown in Table 2 are all based on “% by weight” as active raw materials.

TABLE 2

| Ingredients | Ex. 3 |
|---|---|
| Water | QS 100 |
| Arginine | QS PH7 |
| Cocobetaine | 2.7 |
| Rhamnolipids | 3 |
| Capryloyl Salicylic Acid | 0.3 |
| Phenoxyethanol | 0.3 |
| Glycerin | 5 |
| 1,3-Butylene glycol | 5 |

The composition according to Example 3 also exhibits a good effect of removing or decreasing keratinous plugs, and thus is very suitable as cosmetic compositions for caring for, conditioning, and/or cleansing keratinous substances.

### Examples 4 to 8

Homogeneous solution compositions according to Examples 4 to 8 were also prepared, which have preferred formulations for cosmetic products, by mixing the ingredients shown in Table 3 below. The numerical values for the amounts of the ingredients shown in Table 3 are all based on “% by weight” as active raw materials.

TABLE 3

| Ingredients | Ex. 4 | Ex. 5 | Ex. 6 | Ex. 7 | Ex. 8 |
|---|---|---|---|---|---|
| Water | QS 100 | QS 100 | QS 100 | QS 100 | QS 100 |
| Arginine | QS pH7 | QS pH7 | QS PH7 | QS pH7 | QS PH7 |
| Cocobetaine | 2.7 | 2.7 | 2.7 | 2.7 | 2.7 |
| Sophorolipids | — | — | 3 | 3 | — |
| Rhamnolipids | 3 | 3 | — | — | 3 |
| Salicylic Acid | 0.2 | — | 0.2 | 1 | 0.2 |
| Ethanol | — | 1 | — | 1 | — |
| Triethyl Citrate | — | — | — | — | 2 |
| Capryloyl Salicylic Acid | 0.3 | 0.3 | 0.3 | 0.3 | 0.3 |
| Phenoxyethanol | 0.3 | 0.3 | 0.3 | 0.3 | 0.3 |
| Glycerin | 5 | 5 | 5 | 5 | 5 |
| 1,3-Butylene glycol | 5 | 5 | 5 | 5 | 5 |

The compositions according to Examples 4 to 8 also exhibit a good effect of removing or decreasing keratinous plugs, and thus are very suitable as cosmetic compositions for caring for, conditioning, and/or cleansing keratinous substances.

Regarding those compositions according to Example 3 to 8, they may be packaged in various form, in particular in foam-pump bottles.

1. A cosmetic composition comprising:
(a) at least one glycolipid; and
(b) at least one salicylic acid derivative of formula (I): 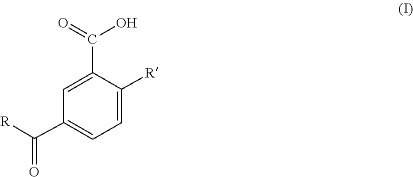
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 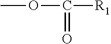
in which R₁ denotes a linear or branched, saturated or unsaturated aliphatic chain containing 1 to 18 carbon atoms;
and also salts thereof derived from an inorganic or organic base.

2. The composition according to claim 1, wherein the (a) glycolipid is selected from rhamnolipids and sophorolipids.

3. The composition according to claim 1, wherein the (b) salicylic acid derivative is represented by formula (I) in which the radical R denotes a linear, branched or cyclic, saturated aliphatic chain containing from 2 to 22 carbon atoms.

4. The composition according to claim 1, wherein the (b) salicylic acid derivative is represented by formula (I) in which the R radical denotes a linear or branched alkyl or alkenyl group, containing 2 or more carbon atoms, and/or 22 or less carbon atoms.

5. The composition according to claim 1, wherein the (b) salicylic acid derivative is represented by formula (I) in which R′ denotes a hydroxyl group.

6. The composition according to claim 1, wherein the amount of the (a) glycolipid(s) is 0.1% by weight or more, and is 10% by weight or less, relative to the total weight of the composition.

7. The composition according to claim 1, wherein the amount of the (b) salicylic acid derivative(s) is 0.01% by weight or more, and is 5% by weight or less, relative to the total weight of the composition.

8. The composition according to claim 1, wherein the weight ratio of the (a) glycolipid(s) to the (b) salicylic acid derivative(s) in the composition is 1 or more.

9. The composition according to claim 1, wherein the composition is in the form of a solution.

10. The composition according to claim 1, wherein the composition further includes water in an amount of 60% by weight or more, relative to the total weight of the composition.

11. The composition according to claim 1, wherein the composition is a cosmetic composition for caring for, conditioning, and/or cleansing keratinous substances.

12. A non-therapeutic cosmetic process for caring for, conditioning, and/or cleansing keratinous substances, comprising:
applying onto the keratinous substances a composition comprising:
(a) at least one glycolipid; and
(b) at least one salicylic acid derivative of formula (I): 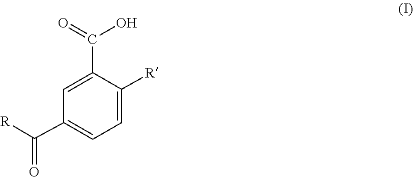
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 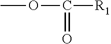
in which R₁ denotes a linear or branched, saturated or unsaturated aliphatic chain containing 1 to 18 carbon atoms;
and also salts thereof derived from an inorganic or organic base.

13. A method comprising:
caring for, conditioning, and/or cleansing keratinous substances with a combination of
(a) at least one glycolipid; and
(b) at least one salicylic acid derivative of formula (I): 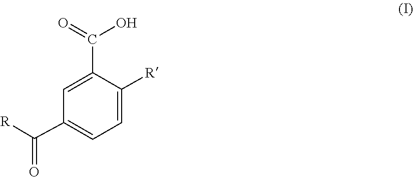
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 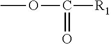
in which R₁ denotes a linear or branched, saturated or unsaturated aliphatic chain containing 1 to 18 carbon atoms;
and also salts thereof derived from an inorganic or organic base.
